(19) United States
(12) Patent Application Publication
(10) Pub. No.: US 2004/0110218 A1
(43) Pub. Date: Jun. 10, 2004

(54) BMOG, a novel protein member of the myelin-oligodendrocyte glycoprotein family and its use for immunomodulatory purposes

(76) Inventor: Jeffrey Browning, Brookline, MA (US)

Correspondence Address: FISH & NEAVE, 1251 AVENUE OF THE AMERICAS, 50TH FLOOR, NEW YORK, NY, 10020-1105, US

(21) Appl. No.: 10/696,259

(22) Filed: Oct. 28, 2003

Related U.S. Application Data
(63) Continuation of application No. 09/560,855, filed on Apr. 28, 2000, now abandoned.
Application No. 09/560,855 is a continuation of application No. PCT/US98/23826, filed on Nov. 6, 1998.
(60) Provisional application No. 60/064,761, filed on Nov. 7, 1997.

(51) Int. Cl. C12Q 1/68; C07H 21/04; C07K 14/47

(52) U.S. Cl. 435/6; 435/69.1; 435/320.1; 435/325; 530/395; 536/23.5

(57) ABSTRACT

BMOG, A Novel Protein Member of the Myelin-Oligodendrocyte Glycoprotein Family which is expressed by germinal center B cells and Its Use For Immunomodulatory Purposes

### RELATED APPLICATIONS

This is a continuation of PCT/US98/23826, filed on 5 Nov. 1998 as a continuation of prior U.S. provisional Serial No. 60/064,761, filed 7 Nov. 1997. The entire disclosure of each of the aforesaid patent applications are incorporated herein by reference.

### SUMMARY OF THE INVENTION

The present invention relates to the use of a novel protein, BMOG (B cell myelin-oligodendrocyte glycoprotein) which is expressed by germinal center B cells. This protein may have immunoregulatory functions and soluble or chimeric fusion proteins of BMOG may be used to regulate the immune system in autoimmune or inflammatory disease.

### BACKGROUND OF THE INVENTION

MOG or myelin-oligodendrocyte glycoprotein is a member of a family of proteins that includes butyrophilin, a glycoprotein expressed in lactating tissue. MOG and butyrophilin are considered to be part of a larger group called the B7 family (1, 2). These proteins have one or two immunoglobulin superfamily domains followed by one or two transmembrane domains. The intracellular domain is either very short such as in MOG, or longer as in the case of butyrophilin, and resembles a B30 ring finger domain. MOG is the best characterized member of the family of single Ig domain members and is a component of myelin. In experimental allergic encephalitis (EAE), immunization of mice with MOG itself or a peptide from the extracellular region of the molecule induces myelin degeneration resulting in a motor neuron deficit (3-5). The disease in this case resembles that in human multiple sclerosis (MS) and therefore murine EAE is considered a model of MS (6). In MS, some initiating event for which viral infections have been postulated, leads to recognition of several proteins in the myelin including, MOG, myelin basic protein (MBP) and proteolipid protein (PLP). MOG is a glycoprotein and has an unusual oligosaccharide epitope on its extracellular domain, i.e. the NHK domain (7) and its role in the nervous system is unclear. The two Ig domain members of this family are the B7-1 and B7-2 ligands for the CTLA-4 and CD28 receptors. The B7 ligands are critical components of T cell activation, and binding to their receptors initiates a co-stimulatory signal (8). Since the B7 proteins play a critical costimulatory role, it is likely that other members of this family will signal through specific receptors.

The immune system reacts to foreign matter by making antibodies and by using various cell types to directly attack it. The antibody response is made by B cells. Antigen presenting cells capture antigen and present it to the B and T cells. The B cells become activated and differentiate into antibody producing cells. Some B cells can migrate into specialized regions of the lymphoid tissues and refine the type of antibody made into a higher affinity form, and some of these B cells further differentiate into specialized B cells called memory B cells. Memory B cells persist for a long time awaiting reemergence of the foreign matter at which time they will activate and proliferate. Therefore, the generation of memory B cells is a critical part of the long term defense strategy of the immune system. Both the process of increasing antibody affinity, called affinity maturation, and the generation of memory B cells occurs in a specialized region in the B cell rich region of the lymph nodes and spleen called a germinal center or secondary follicle. B cells in the germinal center can be recognized in a number of ways, but histologically, they can be identified by their expression of a unique sugar structure that binds the lectin peanut agglutinin. The germinal center contains several cell types and the unique geometry of this region is believed to provide the optimal environment for the generation of memory B cells. B-cell memory is a critical component of an effective vaccine and the general host defense system. In antibody-based autoimmune disease where the immune system has generated antibodies against “self” rather than just against foreign agents the memory B cells are critical in perpetuating the disease state (6).

### SUMMARY

The present invention is directed to a new member of the MOG family called BMOG which presents several possible therapeutic applications. The inventor has discovered this new protein and defining its protein sequence as well as the DNA sequence encoding it. The claimed invention may be used to identify new diagnostics for immune disorders especially those involving antibody defects. Moreover, the inventor defines how a soluble version of the protein could be formed that could either serve to activate or block immunoregulatory events allowing the manipulation of the immune system in immunological disease.

Thus, to achieve these and other advantages, and in accordance with the purpose of the invention, as embodied and broadly described herein, the invention includes DNA sequences encoding human BMOG which may assume three different forms depending on the splicing point for the last exon. These three variations merely vary the intracellular portion of the molecule. These three cDNA sequences are defined as SEQ. ID NOS. 1-3. The invention includes DNA sequences enabling one to express the protein either in a full length form or as a truncated soluble molecule. In the latter case, the molecule may be coupled to other proteins such as an immunoglobulin Fc domain to confer favorable properties such as a long serum half-life.

In other embodiments, the invention relates to the production of recombinant forms of BMOG or fragments thereof. The recombinant proteins may either act as soluble ligands and induce an immunological response or in a soluble state they may block the interaction between cell surface BMOG and a receptor protein. As such they would have immunomodulatory activities that are pharmacologically useful to manipulate the immune system in immunological disease. The proteins may form the basis for diagnostic tests of antibody production to BMOG in disease states. The proteins can be used to create specific antibodies and these anti-BMOG antibodies are also a component of this invention. These antibodies may either block immunological responses or may be incorporated into diagnostic tests. Antibodies can also cross-link antigens and make them more effective in a polyvalent state and therefore could activate an immunological response.

In yet other embodiments, the invention relates to methods of gene therapy using the BMOG gene.

The pharmacological preparations of the invention may, optionally, include pharmaceutically acceptable carriers, adjuvants, fillers or other pharmaceutical compositions and may be administered in any of the numerous forms or routes known in the art.

It is also understood that both the foregoing general description and the following detailed description are exemplary and explanatory and are intended to provide further explanation of the invention as claimed.

The accompanying drawings are included to provide a further understanding of the invention and are incorporated in and constitute a part of this specification, illustrate several embodiments of the invention and together with the description serve to explain the principles of the invention.

### BRIEF DESCRIPTION OF THE DRAWINGS

FIG. 1 is a schematic illustration of the protein structure comparing BMOG and MOG. FIG. 2 delineates the amino acid sequence of BMOG with each of the 3 possible C terminal splice variants. Various portions of the molecule are identified.

FIG. 3 shows an alignment of amino acid sequences of human BMOG, human, rat and mouse MOG (hu- rat- and mo-MOG), chicken B-G gene (ch B-G), human and bovine butyrophilin (hu-Bu and bov-Bu) and human B7-1 and human B7-2. Only the first Ig domain of butyrophilin, B7-1 and B7-2 is shown. Underlined regions shows the approximate transmembrane region in BMOG and MOG.

FIG. 4: Northern analysis of BMOG expression in various human tissues and cell lines revealing the predominant expression in the lymphoid system.

FIG. 5: A. Expression of a recombinant soluble form of human BMOG comprising the extracellular domain. SDS-PAGE analysis of the metal-chelate affinity purified protein.

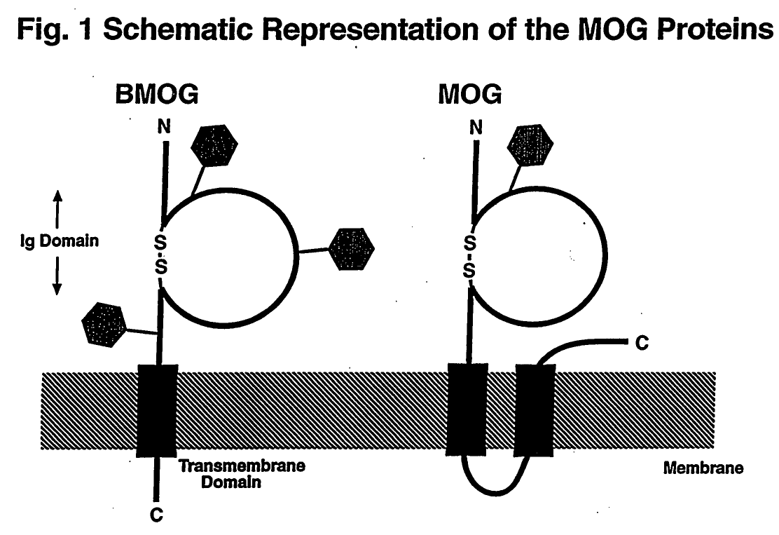

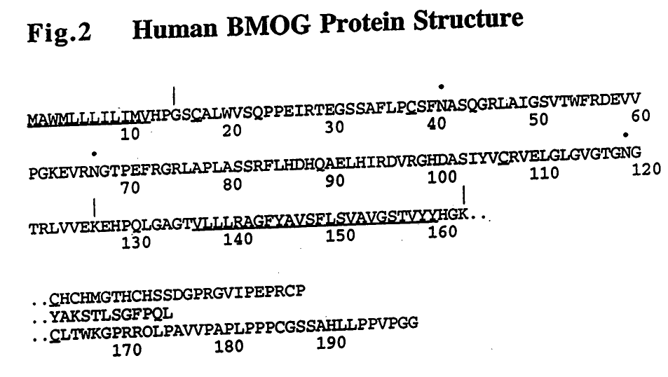

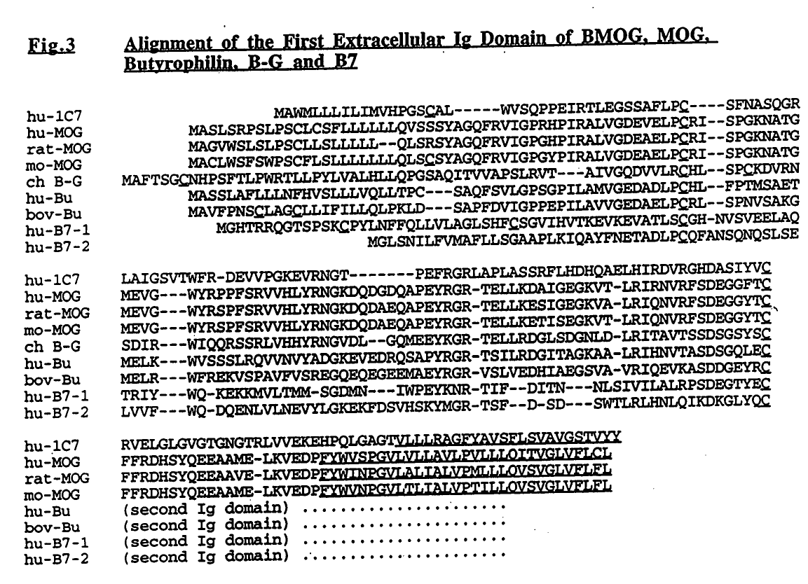

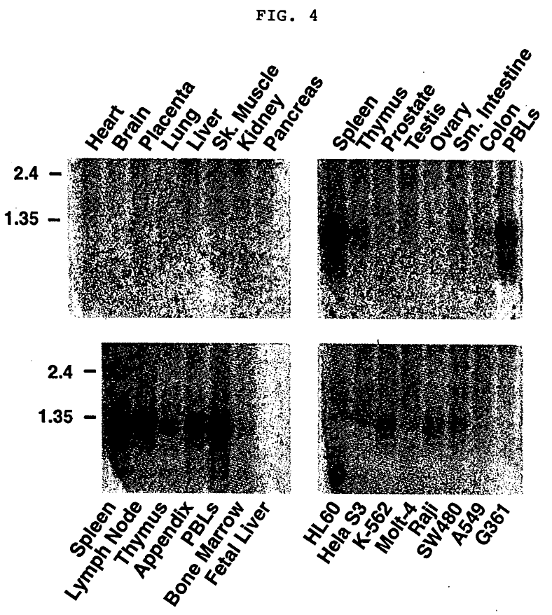

B. Expression of a recombinant soluble form of human BMOG-mouse Ig fusion protein comprising the extracellular domain coupled to the CH2+CH3 domains of mouse IgG1. SDS-PAGE analysis of the protein A affinity purified protein.

### DETAILED DESCRIPTION

### A. Definition

“Homologous”, as used herein, refers to the sequence similarity between sequences of molecules being compared. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared×100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

A “purified preparation” or a “substantially pure preparation” of a polypeptide, as used herein, means a polypeptide that has been separated from other proteins, lipids, and nucleic acids with which it naturally occurs. Preferably, the polypeptide is also separated from other substances, e.g., antibodies, matrices, etc., which are used to purify it.

“Transformed host” as used herein is meant to encompass any host with stably integrated sequence, i.e. BMOG or BMOG sequence, introduced into its genome or a host possessing sequence, i.e. BMOG encoding episomal elements.

A “treatment”, as used herein, includes any therapeutic treatment, e.g., the administration of a therapeutic agent or substance, e.g., a drug.

A “substantially pure nucleic acid”, e.g., a substantially pure DNA, is a nucleic acid which is one or both of: (1) not immediately contiguous with either one or both of the sequences, e.g., coding sequences, with which it is immediately contiguous (i.e., one at the 5′ end and one at the 3′ end) in the naturally-occurring genome of the organism from which the nucleic acid is derived; or (2) which is substantially free of a nucleic acid sequence with which it occurs in the organism from which the nucleic acid is derived. The term includes, for example, a recombinant DNA which is incorporated into a vector, e.g., into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment) independent of other DNA sequences. Substantially pure DNA also includes a recombinant DNA which is part of a hybrid gene encoding BMOG.

The terms “peptides”, “proteins”, and “polypeptides” are used interchangeably herein. “

Biologically active” as used herein, means having an in vivo or in vitro activity which may be performed directly or indirectly. Biologically active fragments of BMOG may have, for example, 70% amino acid homology with the active site of the BMOGs, more preferably at least 80%, and most preferably, at least 90% homology. Identity or homology with respect to the BMOGs is defined herein as the percentage of amino acid residues in the candidate sequence which are identical to the BMOG residues in SEQ. ID. NOS. 4, 5 or 6.

Compounds and Compositions of the Invention:

A. Nucleic Acids

As described herein, one aspect of the invention features an isolated, substantially pure (or recombinant) nucleic acid which includes a nucleotide sequence encoding BMOG, such as the DNA sequences described in SEQ ID. NO's 1-3. SEQ ID NO's refer to three C-terminal splicing variants that have been identified, and they encode the protein sequences given in SEQ ID NO's 4-6. To date, one protein sequence lacking the 5′N terminal exon and containing one possible splice variant has been described and the gene was placed next to the B144 gene in the human MHC, i.e. in the class III region close to the TNF cytokine locus (9). Three EST entries are known to exist describing parts of the cDNA, however, there is no complete cDNA structure in the known in the database. Using genomic sequence which we had previously generated from cosmid clones which spanned the TNF/B144 locus, it was possible to completely and uniquely define the cDNA structure of the three splice variants. As only these three EST entries which were obtained from a FACS sorted germinal center B cell library are found in the database, it is likely that BMOG transcripts are relatively limited to germinal center B cells indicating an immunological function.

Using a fragment of the BMOG DNA, a northern blot was probed to determine BMOG expression patterns. Expression was observed (figure) only in spleen, peripheral blood lymphocytes, (PBL's), lymph nodes, thymus and appendix indicating selective localization to the immune system and in agreement with the presence of RNA in the FACS sorted germinal center B cell library in which EST's were detected.

The DNA sequences of the invention can be employed to produce the claimed BMOG peptides on expression in various prokaryotic and eukaryotic hosts transformed with them. These peptides may be used in anti-cancer, and immunoregulatory applications. In general, this comprises the steps of culturing a host transformed with a DNA molecule containing the sequence encoding BMOG or a fragment thereof, operatively-linked to an expression control sequence.

The DNA sequences and recombinant DNA molecules of the present invention can be expressed using a wide variety of host/vector combinations. For example, useful vectors may consist of segments of chromosomal, non-chromosomal or synthetic DNA sequences. The expression vectors of the invention are characterized by at least one expression control sequence that may be operatively linked to one of the BMOG DNA sequence inserted in the vector, in order to control and to regulate the expression of the DNA sequence.

Furthermore, within each expression vector, various sites may be selected for insertion of a BMOG sequence of the invention. The sites are usually designated by a restriction endonuclease which cuts them, and these sites and endonucleases are well recognized by those skilled in the art. It is of course to be understood that an expression vector useful in this invention need not have a restriction endonuclease site for insertion of the desired DNA fragment. Instead, the vector may be cloned to the fragment by alternate means. The expression vector, and in particular the site chosen therein for insertion of a selected DNA fragment, and its operative linking therein to an expression control sequence, is determined by a variety of factors. These factors include, but are not limited to, the size of the protein to be expressed, the susceptibility of the desired protein to proteolytic degradation by host cell enzymes, number of sites susceptible to a particular restriction enzyme, contamination or binding of the protein to be expressed by host cell proteins which may prove difficult to remove during purification. Additional factors which may be considered include expression characteristics such as the location of start and stop codons relative to the vector sequences, and other factors which will be recognized by those skilled in the art. The choice of a vector and insertion site for the claimed DNA sequences is determined by a balancing of these factors, not all selections being equally effective for a desired application. However, it is routine for one skilled in the art to analyze these parameters and choose an appropriate system depending on the particular application.

One skilled in the art can readily make appropriate modifications to the expression control sequences to obtain higher levels of protein expression, i.e. by substitution of codons, or selecting codons for particular amino acids that are preferentially used by particular organisms, to minimize proteolysis or to alter glycosylation composition. Likewise, cysteines may be changed to other amino acids to simplify production, refolding or stability problems.

Thus, not all host/expression vector combinations function with equal efficiency in expressing the DNA sequences of this invention. However, a particular selection of a host/expression vector combination may be made by those of skill in the art. Factors one may consider include, for example, the compatibility of the host and vector, toxicity to the host of the proteins encoded by the DNA sequence, ease of recovery of the desired protein, expression characteristics of the DNA sequences and expression control sequences operatively linked to them, biosafety, costs and the folding, form or other necessary post-expression modifications of the desired protein.

Another aspect of the invention relates to the use of the isolated nucleic acid encoding either BMOG in “antisense” therapy. As used herein, “antisense” therapy refers to administration or in situ generation of oligonucleotides or their derivatives which specifically hybridize under cellular conditions with the cellular mRNA and/or DNA encoding the BMOG of interest, so as to inhibit expression of the encoded protein, i.e. by inhibiting transcription and/or translation. The binding may be by conventional base pair complementarity, or, for example, in the case of binding to DNA duplexes, through specific interactions in the major groove of the double helix. In general, “antisense” therapy refers to a range of techniques generally employed in the art, and includes any therapy which relies on specific binding to oligonucleotide sequences.

An antisense construct of the present invention can be delivered, for example, as an expression plasmid, which, when transcribed in the cell, produces RNA which is complementary to at least a portion of the cellular mRNA which encodes BMOG. Alternatively, the antisense construct can be an oligonucleotide probe which is generated ex vivo. Such oligonucleotide probes are preferably modified oligonucleotides which are resistant to endogenous nucleases, and are therefor stable in vivo. Exemplary nucleic acids molecules for use as antisense oligonucleotides are phosphoramidates, phosphothioate and methylphosphonate analogs of DNA (See, e.g., U.S. Pat. Nos. 5,176,996; 5,264,564; and 5,256,775). Additionally, general approaches to constructing oligomers useful in antisense therapy have been reviewed, for example, by Van Der Krol et al., (1988) Biotechniques 6:958-976; and Stein et al. (1988) Cancer Res 48: 2659-2668, specifically incorporated herein by reference.

This invention also relates to the use of the DNA sequences disclosed herein to express these BMOG polypeptides under abnormal conditions, i.e. in a gene therapy setting. BMOG may be expressed in various tissues under the direction of promoters appropriate for such applications. Such expression could enhance immune responses or directly affect the survival of the tissue.

In addition, the compounds of the invention include sequences which include the above sequences, or are derivatives of one of these sequences. The invention also includes vectors, liposomes and other carrier vehicles which encompass one of these sequences or a derivative of one of these sequences. The invention also includes the proteins transcribed and translated from these DNA sequences, their derivatives and variants, as discussed in more detail below.

### B. BMOG and Amino Acid Sequences Therefor

The BMOGs of the invention, as discussed above, and the polypeptide, fragments or homologs derived therefrom have wide therapeutic and diagnostic applications. BMOG is a protein in the Ig superfamily and is related to MOG, a gene in the chicken MHC called B-G, and a protein associated with lactating tissues called butyrophilin. There is a weaker relationship between BMOG and the B7 BMOGs, B7-1 and B7-2. By analogy with the B7 system, it is likely that there are receptors for these proteins, i.e. MOG and BMOG although at this time, nothing is currently known. BMOG is present primarily in the spleen, and lymph nodes, in germinal center B cells, strongly indicating a regulatory role in the immune system.

The novel polypeptides of the invention specifically interact with a receptor, which has not yet been identified. However, the peptides and methods disclosed herein enable the identification of receptors which specifically interact with the claimed BMOGs or fragments thereof. The claimed invention in certain embodiments includes polypeptides derived from BMOG which have the ability to bind to BMOG receptors.

Fragments of BMOG can be produced in several ways, e.g., recombinantly, by PCR, proteolytic digestion or by chemical synthesis. Internal or terminal fragments of a polypeptide can be generated by removing one or more nucleotides from one end or both ends of a nucleic acid which encodes the polypeptide. Expression of the mutagenized DNA produces polypeptide fragments. Digestion with “end-nibbling” endonucleases can thus generate DNAs which encode a variety of fragments. DNAs which encode fragments of a protein can also be generated by random shearing, restriction digestion or a combination of the above discussed methods.

Polypeptide fragments can also be chemically synthesized using techniques known in the art such as conventional Merrifield solid phase f-moc or t-boc chemistry. For example, peptides and DNA sequences of the present invention may be arbitrarily divided into fragments of desired length with no overlap of the fragment, or divided into overlapping fragments of a desired length. Methods such as these are described in more detail below.

Soluble forms of BMOG can often signal effectively and hence can be administered as a drug which mimics the natural membrane form. The BMOGs claimed herein may be naturally secreted, however, if not, one can reengineer the gene to force secretion. To create a soluble secreted form of BMOG, one can remove at the DNA level the N-terminus transmembrane regions, and some portion of the stalk region, and replace them with a type I leader or alternatively a type II leader sequence that will allow efficient proteolytic cleavage in the chosen expression system. A skilled artisan could vary the amount of the stalk region retained in the secretion expression construct to optimize both receptor binding properties and secretion efficiency. For example, the constructs containing all possible stalk lengths, i.e. N-terminal truncations, could be prepared such that proteins starting at amino acids 81 to 139 would result. The optimal length stalk sequence would result from this type of analysis.

Since BMOG is a member of the Ig superfamily, one can readily make soluble fusion proteins with other Ig family members, e.g. immunoglobulins, to create molecules that are soluble, easily purified and will have longer half-lives in the blood. The soluble forms of BMOG described herein can serve as the basis for the identification of a BMOG receptor.

The BMOG polypeptides and homologs thereof produced by hosts transformed with the sequences of the invention, as well as native BMOG purified by the processes of this invention, or produced from the claimed amino acid sequences, are useful in a variety of compositions and methods for anticancer, antitumor and immunoregulatory applications. They are also useful in therapy and methods directed to other diseases.

### C. Generation of Antibodies Reactive with BMOG

The invention also includes antibodies specifically reactive with the claimed BMOG or their receptors. Anti-protein/anti-peptide antisera or monoclonal antibodies can be made by standard protocols.(See, for example, Antibodies: A Laboratory Manual ed. by Harlow and Lane (Cold Spring Harbor Press: 1988), incorporated herein by reference). A mammal such as a mouse, a hamster or rabbit can be immunized with an immunogenic form of the peptide. Techniques for conferring immunogenicity on a protein or peptide include conjugation to carriers, or other techniques, well known in the art.

An immunogenic portion of the claimed BMOGs or their receptors can be administered in the presence of an adjuvant. The progress of immunization can be monitored by detection of antibody titers in plasma or serum. Standard ELISA or other immunoassays can be used with the immunogen as antigen to assess the levels of antibodies.

In a preferred embodiment, the subject antibodies are immunospecific for antigenic determinants of BMOG, or its receptors, e.g. antigenic determinants of a polypeptide of SEQ. ID. NO.: 4, 5 or 6, or a closely related human or non-human mammalian homolog (e.g. 70, 80 or 90 percent homologous, more preferably at least 95 percent homologous). In yet a further preferred embodiment of the present invention, the anti-BMOG or anti-BMOG-receptor antibodies do not substantially cross react (i.e. react specifically) with a protein which is e.g., less than 80 percent homologous to SEQ. ID. NO. 4, 5 or 6 preferably less than 90 percent homologous with SEQ. ID. NO.: 4, 5 or 6; and, most preferably less than 95 percent homologous with SEQ. ID. NO.: 4, 5 or 6. By “not substantially cross react”, it is meant that the antibody has a binding affinity for a non-homologous protein which is less than 10 percent, more preferably less than 5 percent, and even more preferably less than 1 percent, of the binding affinity for a protein of SEQ. ID. NO. 4, 5 or 6.

The term antibody as used herein is intended to include fragments thereof which are also specifically reactive with BMOG, or their receptors. Antibodies can be fragmented using conventional techniques and the fragments screened for utility in the same manner as described above for whole antibodies. For example, F(ab′)₂ fragments can be generated by treating antibody with pepsin. The resulting F(ab)₂ fragment can be treated to reduce disulfide bridges to produce Fab′ fragments. The antibodies of the present invention are further intended to include biospecific and chimeric molecules having anti-BMOG or anti-BMOG-receptor activity. Thus, both monoclonal and polyclonal antibodies (Ab) directed against BMOG, BMOG receptors, and antibody fragments such as Fab′ and F(ab′)₂, can be used to block the action of the BMOG and their respective receptor.

Various forms of antibodies can also be made using standard recombinant DNA techniques. (Winter and Milstein, Nature 349: 293-299 (1991) specifically incorporated by reference herein.) For example, chimeric antibodies can be constructed in which the antigen binding domain from an animal antibody is linked to a human constant domain (e.g. Cabilly et al., U.S. Pat. No. 4,816,567, incorporated herein by reference). Chimeric antibodies may reduce the observed immunogenic responses elicited by animal antibodies when used in human clinical treatments.

In addition, recombinant “humanized antibodies” which recognize BMOG s or their receptors can be synthesized. Humanized antibodies are chimeras comprising mostly human IgG sequences into which the regions responsible for specific antigen-binding have been inserted. Animals are immunized with the desired antigen, the corresponding antibodies are isolated, and the portion of the variable region sequences responsible for specific antigen binding are removed. The animal-derived antigen binding regions are then cloned into the appropriate position of human antibody genes in which the antigen binding regions have been deleted. Humanized antibodies minimize the use of heterologous (i.e. inter species) sequences in human antibodies, and thus are less likely to elicit immune responses in the treated subject.

Construction of different classes of recombinant antibodies can also be accomplished by making chimeric or humanized antibodies comprising variable domains and human constant domains (CH1, CH2, CH3) isolated from different classes of immunoglobulins. For example, antibodies with increased antigen binding site valencies can be recombinantly produced by cloning the antigen binding site into vectors carrying the human: chain constant regions. (Arulanandam et al., J. Exp. Med., 177: 1439-1450 (1993), incorporated herein by reference.)

In addition, standard recombinant DNA techniques can be used to alter the binding affinities of recombinant antibodies with their antigens by altering amino acid residues in the vicinity of the antigen binding sites. The antigen binding affinity of a humanized antibody can be increased by mutagenesis based on molecular modeling. (Queen et al., Proc. Natl. Acad. Sci. 86: 10029-33 (1989) incorporated herein by reference.

### D. Generation of Analogs: Production of Altered DNA and Peptide Sequences

Analogs of the claimed BMOG can differ from the naturally occurring BMOG in amino acid sequence, or in ways that do not involve sequence, or both. Non-sequence modifications include in vivo or in vitro chemical derivatization of the BMOGs. Non-sequence modifications include, but are not limited to, changes in acetylation, methylation, phosphorylation, carboxylation or glycosylation.

Preferred analogs include BMOG or biologically active fragments thereof, whose sequences differ from the sequence given in SEQ. ID NOS. 4, 5 or 6, by one or more conservative amino acid substitutions, or by one or more non-conservative amino acid substitutions, deletions or insertions which do not abolish the activity of BMOG. Conservative substitutions typically include the substitution of one amino acid for another with similar characteristics, e.g. substitutions within the following groups: valine, glycine; glycine, alanine; valine, isoleucine, leucine; aspartic acid, glutamic acid; asparagine, glutamine; serine, threonine; lysine, arginine; and, phenylalanine, tyrosine.

TABLE 1
CONSERVATIVE AMINO ACID REPLACEMENTS

| for amino Acid | code | replace with any of: |
|---|---|---|
| Alanine | A | D-Ala, Gly, Beta-Ala, L- |
| | | Cys, D-Cys |
| Arginine | R | D-Arg, Lys, D-Lys, homo- |
| | | Arg, D-homo-Arg, Met, |
| | | Ile, D-Met, D-Ile, Orn, D- |
| | | Orn |
| Asparagine | N | D-Asn, Asp, D-Asp, Glu, |
| | | D-Glu, Gln, D-Gln |
| Aspartic Acid | D | D-Asp, D-Asn, Asn, Glu, |
| | | D-Glu, Gln, D-Gln |
| Cysteine | C | D-Cys, S-Me-Cys, Met, D- |
| | | Met, Thr, D-Thr |
| Glutamine | Q | D-Gln, Asn, D-Asn, Glu, |
| | | D-Glu, Asp, D-Asp |
| Glutamic Acid | E | D-Glu, D-Asp, Asp, Asn, |
| | | D-Asn, Gln, D-Gln |
| Glycine | G | Ala, D-Ala, Pro, D-Pro, - |
| | | Ala, Acp |
| Isoleucine | I | D-Ile, Val, D-Val, Leu, D- |
| | | Leu, Met, D-Met |
| Leucine | L | D-Leu, Val, D-Val, Leu, |
| | | D-Leu, Met, D-Met |
| Lysine | K | D-Lys, Arg, D-Arg, |
| | | Homo-arg, D-homo-Arg, |
| | | Met, D-Met, Ile, D-Ile, |
| | | Orn, D-Orn |
| Methionine | M | D-Met, S-Me-Cys, Ile, D- |
| | | Ile, Leu, D-Leu, Val, D- |
| | | Val |
| Phenylalanine | F | D-Phe, Tyr, D-Thr, L- |
| | | Dopa, His, D-His, Trp, D- |
| | | Trp, Trans-3, 4 or 5- |
| | | phenylproline, cis-3, 4, or |
| | | 5-phenylproline |
| Proline | P | D-Pro, L-I-thoazolidine-4- |
| | | carboxylic acid, D-or L-1- |
| | | oxazolidine-4-carboxylic |
| | | acid |
| Serine | S | D-Ser, Thr, D-Thr, allo- |
| | | Thr, Met, D-Met, Met(O), |
| | | D-Met(O), L-Cys, D-Cys |
| Threonine | T | D-Thr, Ser, D-Ser, allo- |
| | | Thr, Met, D-Met, Met(O), |
| | | D-Met(O), Val, D-Val |
| Tyrosine | Y | D-Tyr, Phe, D-Phe, L- |
| | | Dopa, His, D-His |
| Valine | V | D-Val, Leu, D-Leu, Ile, D- |
| | | Ile, Met, D-Met |

Useful methods for mutagenesis include PCR mutagenesis and saturation mutagenesis as discussed in more detail below. A library of random amino acid sequence variants can also be generated by the synthesis of a set of degenerate oligonucleotide sequences.

PCR Mutagenesis

In PCR mutagenesis, reduced Taq polymerase fidelity can be used to introduce random mutations into a cloned fragment of DNA (Leung et al., 1989, Technique 1:11-15). This is a very powerful and relatively rapid method of introducing random mutations. The DNA region to be mutagenized can be amplified using the polymerase chain reaction (PCR) under conditions that reduce the fidelity of DNA synthesis by Taq DNA polymerase, e.g., by using a dGTP/dATP ratio of five and adding Mn²⁺ to the PCR reaction. The pool of amplified DNA fragments can be inserted into appropriate cloning vectors to provide random mutant libraries.

Saturation Mutagenesis

Saturation mutagenesis allows for the rapid introduction of a large number of single base substitutions into cloned DNA fragments (Mayers et al., 1985, Science 229:242). This technique includes generation of mutations, e.g., by chemical treatment or irradiation of single-stranded DNA in vitro, and synthesis of a complimentary DNA strand. The mutation frequency can be modulated by modulating the severity of the treatment, and essentially all possible base substitutions can be obtained. Because this procedure does not involve a genetic selection for mutant fragments both neutral substitutions, as well as of a protein can be prepared by random mutagenesis of DNA which those that alter function, can be obtained. The distribution of point mutations is not biased toward conserved sequence elements.

Degenerate Oligonucleotides

A library of homologs can also be generated from a set of degenerate oligonucleotide sequences. Chemical synthesis of degenerate sequences can be carried out in an automatic DNA synthesizer, and the synthetic genes then ligated into an appropriate expression vector. The synthesis of degenerate oligonucleotides is known in the artⁱ Such techniques have been employed in the directed evolution of other proteinsⁱⁱ.

Non-random or directed, mutagenesis techniques can be used to provide specific sequences or mutations in specific regions. These techniques can be used to create variants which include, e.g., deletions, insertions, or substitutions, of residues of the known amino acid sequence of a protein. The sites for mutation can be modified individually or in series, e.g., by (1) substituting first with conserved amino acids and then with more radical choices depending upon results achieved, (2) deleting the target residue, or (3) inserting residues of the same or a different class adjacent to the located site, or combinations of options 1-3.

Alanine Scanning Mutagenesis

Alanine scanning mutagenesis is a useful method for identification of certain residues or regions of the desired protein that are preferred locations or domains for mutagenesis, Cunningham and Wells (Science 244:1081-1085, 1989) specifically incorporated by reference. In alanine scanning, a residue or group of target residues are identified (e.g., charged residues such as Arg, Asp, His, Lys, and Glu) and replaced by a neutral or negatively charged amino acid (most preferably alanine or polyalanine). Replacement of an amino acid can affect the interaction of the amino acids with the surrounding aqueous environment in or outside the cell. Those domains demonstrating functional sensitivity to the substitutions can then be refined by introducing further or other variants at or for the sites of substitution. Thus, while the site for introducing an amino acid sequence variation is predetermined, the nature of the mutation per se need not be predetermined. For example, to optimize the performance of a mutation at a given site, alanine scanning or random mutagenesis may be conducted at the target codon or region and the expressed desired protein subunit variants are screened for the optimal combination of desired activity.

Oligonucleotide-Mediated Mutagenesis

Oligonucleotide-mediated mutagenesis is a useful method for preparing substitution, deletion, and insertion variants of DNA, see, e.g., Adelman et al., (DNA 2:183, 1983) incorporated herein by reference. Briefly, the desired DNA can be altered by hybridizing an oligonucleotide encoding a mutation to a DNA template, where the template is the single-stranded form of a plasmid or bacteriophage containing the unaltered or native DNA sequence of the desired protein. After hybridization, a DNA polymerase is used to synthesize an entire second complementary strand of the template that will thus incorporate the oligonucleotide primer, and will code for the selected alteration in the desired protein DNA. Generally, oligonucleotides of at least 25 nucleotides in length are used. An optimal oligonucleotide will have 12 to 15 nucleotides that are completely complementary to the template on either side of the nucleotide(s) coding for the mutation. This ensures that the oligonucleotide will hybridize properly to the single-stranded DNA template molecule. The oligonucleotides are readily synthesized using techniques known in the art such as that described by Crea et al. (Proc. Natl. Acad. Sci. USA, 75: 5765[1978]) incorporated herein by reference.

Cassette Mutagenesis

Another method for preparing variants, cassette mutagenesis, is based on the technique described by Wells et al. (Gene, 34:315[1985]) incorporated herein by reference. The starting material can be a plasmid (or other vector) which includes the protein subunit DNA to be mutated. The codon(s) in the protein subunit DNA to be mutated are identified. There must be a unique restriction endonuclease site on each side of the identified mutation site(s). If no such restriction sites exist, they may be generated using the above-described oligonucleotide-mediated mutagenesis method to introduce them at appropriate locations in the desired protein subunit DNA. After the restriction sites have been introduced into the plasmid, the plasmid is cut at these sites to linearize it. A double-stranded oligonucleotide encoding the sequence of the DNA between the restriction sites but containing the desired mutation(s) is synthesized using standard procedures. The two strands are synthesized separately and then hybridized together using standard techniques. This double-stranded oligonucleotide is referred to as the cassette. This cassette is designed to have 3′ and 5′ ends that are comparable with the ends of the linearized plasmid, such that it can be directly ligated to the plasmid. This plasmid now contains the mutated desired protein subunit DNA sequence.

Combinatorial Mutagenesis

Combinatorial mutagenesis can also be used to generate mutants. E.g., the amino acid sequences for a group of homologs or other related proteins are aligned, preferably to promote the highest homology possible. All of the amino acids which appear at a given position of the aligned sequences can be selected to create a degenerate set of combinatorial sequences. The variegated library of variants is generated by combinatorial mutagenesis at the nucleic acid level, and is encoded by a variegated gene library. For example, a mixture of synthetic oligonucleotides can be enzymatically ligated into gene sequences such that the degenerate set of potential sequences are expressible as individual peptides, or alternatively, as a set of larger fusion proteins containing the set of degenerate sequences.

Various techniques are known in the art for screening generated mutant gene products. Techniques for screening large gene libraries often include cloning the gene library into replicable expression vectors, transforming appropriate cells with the resulting library of vectors, and expressing the genes under conditions in which detection of a desired activity, e.g., in this case, binding to BMOGs or their receptors, facilitates relatively easy isolation of the vector encoding the gene whose product was detected. Each of the techniques described below is amenable to high through-put analysis for screening large numbers of sequences created, e.g., by random mutagenesis techniques.

The invention also provides for reduction of the protein binding domains of the claimed polypeptides or their receptors, to generate mimetics, e.g. peptide or non-peptide agents. The peptide mimetics are able to disrupt binding of BMOG with its respective receptor. The critical residues of the BMOG involved in molecular recognition of a receptor polypeptide or of a downstream intracellular protein, can be determined and used to generate the BMOG or its receptor-derived peptidomimetics which competitively or noncompetitively inhibit binding of the BMOG with a receptor. (see, for example, “Peptide inhibitors of human papilloma virus protein binding to retinoblastoma gene protein” European patent applications EP-412,762A and EP-B31,080A), specifically incorporated herein by reference.

In addition to substantially full-length polypeptides comprising the sequences of the invention, the present invention also encompasses biologically active fragments of the polypeptides. A BMOG polypeptide or fragment is biologically active if it exhibits a biologicalactivity of a naturally occurring peptide. such biological activities include the ability to bind to the antibodies directed against epitopes present on naturally occurring BMOG.

In other embodiments, variants with amino acid substitutions s which are less conservative may also result in desired derivatives, e.g., by causing changes in charge, conformation and other biological properties. Such substitutions would include, for example, substitution of hydrophilic residue for a hydrophobic residue, substitution of a c cysteine or proline for another residue, substitution of a residue having a small side chain for a residue having a bulky side chain, or substitution of a residue having a net positive charge for a residue having a net negative charge. When the result of a given substitution cannot be predicted with certainty, the derivatives may be readily assayed according g to the methods disclosed herein to determine the presence or absence of the desired characteristics.

Other variants within the scope of the invention include polypeptides with amino acid sequences having a t least sixty percent homology with SEQ ID NOS 4-6, defined herein. More preferably, the sequence homology is at least eighty, at least ninety percent, or at least ninety-five percent. For the purposes of determining homology, the length of comparison sequences will generally be at least 8 amino acid residues, usually at least 20 amino acid residues. Variants of the compounds of the invention also include any protein which 1) has an amino acid sequence which is at least forty percent homologous to a polypeptide of the invention, and also which, after being placed in an optimal alignment with the sequence of the invention, has at least 80% of its cysteine residues aligned with cysteines in the polypeptides of the invention.

Just as it is possible to replace substituents of the scaffold, it is also possible to substitute functional groups which are bound to the scaffold with groups characterized by similar features. Such modifications do not alter primary sequence. These will initially be conservative, i.e. the replacement group will have approximately the same size, shape, hydrophobicity and charge as the original group. Non-sequence modifications may include, for example, in vivo or in vitro chemical derivatization of portions of naturally occurring BMOG, as well as changes in acetylation, methylation, phosphorylation, carboxylation or glycosylation.

Also included within the scope of the present invention are agents which specifically bind to a polypeptide of the invention. These agents include Ig fusion proteins and antibodies, including single chain, double chain, FAB fragments and others, whether native, humanized, primatized or chimeric. Additional descriptions of these categories of agents are known by those of skill in the art.

### E. Pharmaceutical Compositions

By making available purified and recombinant-BMOG, the present invention provides assays which can be used to screen for drug candidates which are either agonists or antagonists of the normal cellular function, in this case, of BMOG or its receptor(s). In one embodiment, the assay evaluates the ability of a compound to modulate binding between the BMOGs and their receptors. A variety of assay formats will suffice and, in light of the present inventions, will be comprehended by the skilled artisan.

In many drug screening programs which test libraries of compounds and natural extracts, high throughput assays are desirable in order to maximize the number of compounds surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test compound. Moreover, the effects of cellular toxicity and/or bioavailability of the test compound can be generally ignored in the in vitro system, the assay instead being focused primarily on the effect of the drug on the molecular target as may be manifest in an alteration of binding affinity with other proteins or change in enzymatic properties of the molecular target.

Pharmaceutical compositions of the invention may comprise a therapeutically effective amount of BMOG, or its receptor(s), or fragments or mimetics thereof, and, optionally may include pharmaceutically acceptable carriers. Accordingly, this invention provides methods for treatment of cancer, and methods of stimulating, or in certain instances, inhibiting the immune system, or parts thereof by administering a pharmaceutically effective amount of a compound of the invention or its pharmaceutically acceptable salts or derivatives. It should of course by understood that the compositions and methods of this invention can be used in combination with other therapies for various treatments.

The compositions can be formulated for a variety of routes of administration, including systemic, topical or localized administration. For systemic administration, injection is preferred, including intramuscular, intravenous, intraperitoneal, and subcutaneous for injection, the compositions of the invention can be formulated in liquid solutions, preferably in physiologically compatible buffers such as Hank's solution or Ringer's solution. In addition, the compositions may be formulated in solid form and, optionally, redissolved or suspended immediately prior to use. Lyophilized forms are also included in the invention.

The compositions can be administered orally, or by transmucosal or transdermal means. For transmucosal or transdermal administration, penetrants appropriate to the barrier to be permeated are used in the formulation. Such penetrants are known in the art, and include, for example, for transmucosal administration, bile salts, fusidic acid derivatives, and detergents. Transmucosal administration may be through nasal sprays or using suppositories. For oral administration, the compositions are formulated into conventional oral administration forms such as capsules, tablets, and tonics. For topical administration, the compositions of the invention are formulated into ointments, salves, gels, or creams as known in the art.

Preferably the compositions of the invention will be in the form of a unit dose and will be administered one or more times a day. The amount of active compound administered at one time or over the course of treatment will depend on many factors. For example, the age and size of the subject, the severity and course of the disease being treated, the manner and form of administration, and the judgments of the treating physician. However, an effective dose may be in the range of from about 0.005 to about 5 mg/kg/day, preferably about 0.05 to about 0.5 mg/kg/day. One skilled in the art will recognize that lower and higher doses may also be useful.

Gene constructs according to the invention can also be used as a part of a gene therapy protocol to deliver nucleic acids encoding either an agonistic or antagonistic form of a BMOG or BMOG polypeptide.

Expression constructs of the claimed BMOGs can be administered in any biologically effective carrier, e.g., any formulation or composition capable of effectively delivering the gene for the claimed BMOGs to cells in vivo. Approaches include insertion of the gene in viral vectors which can transfect cells directly, or delivering plasmid DNA with the help of, for example, liposomes, or intracellular carriers, as well as direct injection of the gene construct. Viral vector transfer methods are preferred.

A pharmaceutical preparation of the gene therapy construct can consist essentially of the gene delivery system in an acceptable diluent, or can comprise a slow release matrix in which the gene delivery vehicle is imbedded. Alternatively, where the complete gene delivery system can be produced intact from recombinant cells, e.g. retroviral vectors, the pharmaceutical preparation can comprise one or more cells which produce the gene delivery system.

In addition to use in therapy, the oligomers of the invention may be used as diagnostic reagents to detect the presence or absence of the target DNA, RNA or amino acid sequences to which they specifically bind. In other aspects, the claimed invention may be used to evaluate a chemical entity for its ability to interact with, e.g., bind or physically associate with a claimed BMOG, or fragment thereof. The method includes contacting the chemical entity with the BMOG, and evaluating the ability of the entity to interact with the BMOG. Additionally, the BMOGs of the invention can be used in methods of evaluating naturally occurring BMOGs or receptors of these BMOGs, as well as to evaluate chemical entities which associate or bind with receptors of the BMOGs.

In certain aspects, the claimed invention features a method for evaluating a chemical entity for the ability to modulate the interaction between BMOG, BMOG and their respective receptors. The method includes combining a BMOG receptor, and the BMOG under conditions wherein the pair is capable of interacting, adding the chemical entity to be evaluated and detecting the formation or dissolution of complexes. These modulating agents may be further evaluated in vitro, e.g. by testing its activity in a cell free system, and then optionally administering the compound to a cell or animal, and evaluating the effect.

### F. Uses of the Compounds of the Invention

Native and variant BMOG, anti-BMOG antibodies, anti BMOG-receptor antibodies and fusion proteins of BMOG and BMOG receptor may have therapeutic utility in situations where it is desirable to block or to activate the BMOG signaling pathway. In general, the compounds of at the invention can be used to modulate the immune system to obtain a desired result. For example, BMOG signaling can be affected by contacting antibodies to a sample which block or stimulate activation of the BMOG signaling pathway. Certain antibodies will interact with BMOG as agonists, and others, as antagonists. Additional methods encompass imaging of tissues which express BMOG, or immunohistological or preparative methods for antibodies to BMOG.

More preferably, the compounds of the invention can be used as medical therapies for immunological based diseases which involve myelin degeneration. For example, multiple sclerosis.

The compounds of the invention are administered in therapeutically effective amounts, which means that an amount of a compound which produces a medically desirable result or exerts an influence on the particular condition being treated.

The term “subject” as used herein means any mammal to which the compounds of the invention may be administered. Subjects specifically intended for treatment with the methods of the invention include humans, as well as nonhuman primates, sheep, horses, cattle, goats, pigs, dogs, cats, rabbits, guinea pigs, hamsters, gerbils, rats and mice, as well a s the organs, tumors and cells derived or originating from these hosts.

In yet other embodiments, the BMOG genes of the invention can be used in gene therapy. The genes of the invention can be introduced into damaged, or non expressing tissues to stimulate the production of BMOG by the transfected cells, to promote cell growth, and or survival of cells which express BMOG.

In a specific embodiment of a gene therapy method, a gene for BMOG may be introduced into a tissue of choice, preferably a tissue associated with myelin.

Viral or non-viral methods may be used to introduce a gene into a desired tissue. Such methods are known to those skilled in the art. Non-viral methods include for example, electroporation, membrane fusion with liposomes, high velocity bombardment with DNA-coated microporojectiles, incubation with calcium-phosphate DNA precipitate, DEAE-dextran mediated transfection, and direct micro-injection into single cells.

Alternatively, target cells may be transfected with the genes of the invention by direct gene transfer. See e.g., Wolff et al., “Direct Gene Tranfer into Moose Muscle In Vivo”, Science 247: 1465-68, 1990, incorporated herein by reference. In many cases, vector-mediated transfection will be desirable. Any of the methods known in the art for the insertion of polynucleotide sequences into a vector may be used. See, for example, Sambrook et al., Molecular Cloning: A Laboratory Manual, Cold Spring Harbor Laboratory, Cold Spring Harbor, N.Y. 1989, and Ausubel et al., Current Protocols in Molecular Biology, J. Wiley & Sons, NY (1992), both of which are incorporated herein by reference. Promoter activation may be tissue specific or inducible by a metabolic product or administered substance. Such promoters/enhancers include, but are not limited to the native BMOG promoter, the cytomegalovirus immediate-early promoter/enhancer, the late promoter, and other promoters known to those of skill in the art. The BMOG genes may also be introduced by specific viral vectors for use in gene transfer systems which are now well established. See for example: Madzak et al., J. Gene Virol. 73: 1533-36, 1992 (papovirus SV40); Berkner et al., Curr. Top. Microbiol. Immunol., 158: 39-61, 1992 (adenovirus); Hoffman et al., Proc. Nat'l Acad. Sci 92: 10099-10103, 1995 (baculovirus); Moss et al., Curr. Top Microbiol. Immunol., 158: 25-38, 1992 (vaccinia virus); Muzyczka, Curr. Top. Microbiol. Immunol., 158: 97-123 1992 (adeno-associated virus), Margulskee, Curr. Top. Microbiol. Immunol., 158: 67-93, 1992 (herpes simplex virus and Epstein-Barr virus); Miller, Curr top. Microbiol. Immunol., 158: 1-24, 1992 (retrovirus); Brandyopadhyay et al., Mol. Cell. Biol., 4: 749-754, 1984 (retrovirus); Miller et al., Nature, 357: 455-450, 1992 (retro virus), all of which are incorporated herein by reference.

Preferred vectors are DNA viruses that include adenoviruses, baculovirus, herpes viruses and parvovirus.

The compounds of the invention may be administered in any manner which is medically acceptable. This may include injections, by parenteral routes such as intravenous, intravascular, intraarterial, subcutaneous, intramusular, intratumor, intraperitoneal, intraventricular, intraepidural, or others as well as oral, nasal, opthalmic, rectal or topical. Sustained release administration is also specifically included in the invention, by such means as depot injections or erodible implants. Localized delivery is particularly contemplated, by such means as deliver via a catheter to one or more arteries, such as the renal artery or a vessel supplying a localized tumor.

The term pharmaceutically acceptable carrier means one or more organic or inorganic ingredients, natural or synthetic, with which the mutant proto-oncogene or mutant oncoprotein is combine to facilitate its application. A suitable carrier includes sterile saline, although other aqueous and non-aqueous isotonic sterile solutions and sterile suspensions known to be pharmaceutically acceptable are known to those of ordinary skill in the art. An effective amount refers to that amount which is capable of ameliorating or delaying progression of the diseased, degenerative or damaged condition. An effective amount can be determined on an individual basis and will be based, in part, on consideration of the symptoms to be treated and results sought. An effective amount can be determined by one or ordinary skill in the art employing such factors and using no more than routine experimentation.

### G. EXAMPLES

### Example 1

### Preparation of Recombinant Soluble Human BMOG

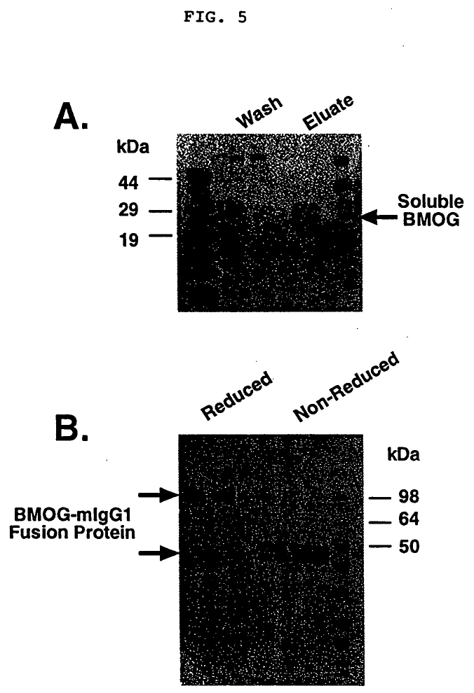

A piece of DNA was prepared by PCR spanning amino acids 1 to 139 of seq 4 and was terminated in an additional aspartate residue and a six histidine tag on the C terminus followed by a stop codon. The reaction used the following primers: 5′AACTGCAGCGGCCGCCATGGCCTGGATGCTGTTG3′ and 5′ATAGTTTAGCGGCCGCTCAGTGATGGTGGTGATGGTGGTCGACTGTACCAGCC CCTAG 3′ which incorporated flanking Not1 sites on the final product. The insert was cloned into an expression vector called CH269 (Chicheportiche et al,. JBC 1997) which would allow for high copy expression in EBNA expressing HEK 293 cells. Upon transfection and purification over a metal chelate affinity resin and elution with imidazole buffer by conventional techniques used to purify Histidine tagged proteins, a roughly 22-29 kDa band was observed representing pure soluble BMOG. The band was larger than predicted for a 146 amino acid protein and it was fuzzy indicating the presence of considerable carbohydrate as expected from the three potential N-linked glycosylation sites (FIG. 5).

### Example 2

### Preparation of a BMOG-immunoglobulin Fusion Protein

Into the above piece of DNA encoding soluble histidine tagged BMOG, a SalI site was incorporated near the N-terminus which when cleaved allowed in frame assembly with the SalI site in a mouse immunoglobulin Fc domain piece yielding a vector with the BMOG extracellular domain as the N-terminus followed by domains CH2 and CH3 of murine IgG1 essentially as described previously (Browning et al 1997, JI 159, 3288). Expression in HEK 293 cells using the above system and protein A affinity purification yielded a recombinant BMOG-Ig fusion protein (FIG. 5). The size of this fusion protein at 45-50 kDa reduced was somewhat larger than expected due to the extensive glycosylation as was seen for the soluble non-Ig fused version. Without reduction, the protein ran at about 100 kDa indicating the formation of a dimer as expected for Ig fusion proteins.

### Example 3

### Isolation of a Receptor Binding to the Claimed BMOGs

BMOGs can be used to identify and clone receptors. With the described BMOG sequences, one could fuse the 5′ end of the extracellular domain of these BMOGs which constitutes the receptor binding sequence to a marker or tagging sequence and then add a leader sequence that will force secretion of the BMOG in any of a number of expression systems. One example of this technology is described by Browning et al., (1996) (JBC 271, 8618-8626) where the LT-β ligand was secreted in such a form. The VCAM leader sequence was coupled to a short myc peptide tag followed by the extracellular domain of the LT-β. The VCAM sequence is used to force secretion of the normally membrane bound LT-β molecule. The secreted protein retains a myc tag on the N-terminus which does not impair the ability to bind to a receptor. Such a secreted protein can be expressed in either transiently transfected Cos cells or a similar system, e.g., EBNA derived vectors, insect cell/baculovirus, picchia etc. The unpurified cell supernatant can be used as a source of the tagged BMOG.

Cells expressing the receptor can be identified by exposing them to the tagged BMOG. Cells with bound BMOG are identified in a FACS experiment by labeling the myc tag with an anti-myc peptide antibody (9E10) followed by phycoerythrin (or a similar label) labeled anti-mouse immunoglobulin. FACS positive cells can be readily identified and would serve as a source of RNA encoding for the receptor. An expression library would then be prepared from this RNA via standard techniques and separated into pools. Pools of clones would be transfected into a suitable host cell and binding of the tagged BMOG to receptor positive transfected cells determined via microscopic examination, following labeling of bound myc peptide tag with an enzyme labeled anti-mouse Ig reagent, i.e. galactosidase, alkaline phosphatase or luciferase labeled antibody. Once a positive pool has been identified, the pool size would be reduced until the receptor encoding cDNA is identified. This procedure could be carried out with either the mouse or human BMOG, as one may more readily lead to a receptor.

It will be apparent to those skilled in the art that various modifications and variations can be made in the novel BMOGs, compositions and methods of the present invention without departing from the spirit or scope of the invention. Thus, it is intended that the present invention cover the modifications and variations of this invention provided that they come within the scope of the appended claims and their equivalents.

### References

1. Henry, J., M.-T. Robouchon, D. Depetris, M.-G. Mattei, C. Offer, R. Tazi-Ahnini, and P. Pontarotti. 1997. Cloning, structural analysis, and mapping of the B30 and B7 multigenic families to the major histocompatibility complex (MHC) and other chromosomal regions. Immunogenetics 46:383-395.

2. Linsley, P. S., R. Peach, P. Gladstone, and J. Bajorath. 1994. Extending the B7 (CD80) family. Protein Sci. 3:1341-1342.

3. Ichikawa, M., T. G. Johns, J. Liu, and C. C. Bernard. 1996. Analysis of the fine B cell specificity during the chronic/relapsing course of a multiple sclerosis-like disease in Lewis rats injected with the encephalitogenic myelin oligodendrocyte glycoprotein peptide 35-55. J Immunol 157:919-26.

4. Kerlero de Rosbo, N., I. Mendel, and A. Ben-Nun. 1995. Chronic relapsing experimental autoimmune encephalomyelitis with a delayed onset and an atypical clinical course, induced in PL/J mice by myelin oligodendrocyte glycoprotein (MOG)-derived peptide: preliminary analysis of MOG T cell epitopes. Eur J Immunol 25:985-93.

5. Mendel, I., N. Kerlero de Rosbo, and A. Ben-Nun. 1995. A myelin oligodendrocyte glycoprotein peptide induces typical chronic experimental autoimmune encephalomyelitis in H-2b mice: fine specificity and T cell receptor V beta expression of encephalitogenic T cells. Eur J Immunol 25:1951-9.

6. Schwarz, R. S. 1993. in Fundamental Immunology. Raven Press, New York.

7. Burger, D., C. C. Bernard, and N. Kerlero de Rosbo. 1993. Human myelin/oligodendrocyte glycoprotein: a new member of the L2/HNK-1 family. J. Neurochem. 61:1822-1827.

8. Lenschow, D. J., T. L. Walunas, and J. A. Bluestone. 1996. CD28/B7 system of T cell costimulation. Ann. Rev. Immunol. 14:233-258.

9. Nalabolu, S. R., H. Shukla, G. Nallur, S. Parimoo, and S. M. Weissman. 1996. Genes in the 220-kb region spanning the TNF cluster in human MHC. Genomics 31:215-222.

What is claimed is:

1. A purified and isolated DNA molecule having a nucleotide sequence comprising a DNA sequence selected from the group consisting of SEQ ID NO 1, SEQ ID NO 2, and SEQ ID NO 3.

2. A DNA molecule coding for an amino acid sequence comprising SEQ ID NO 4, SEQ ID NO 5 or SEQ ID NO 6.

3. A purified and isolated DNA molecule which is at least 80% homologous to SEQ ID NO 1, SEQ ID NO 2, or SEQ ID NO 3.

4. The DNA of claim 3 further characterized by encoding a protein with the biological activity of BMOG.

5. A vector comprising a DNA molecule of claim 1.

6. A prokaryotic or eukaryotic host cell stably transformed or transfected by a vector comprising a DNA molecule of claim 1.

7. A process for the production of a polypeptide having part or all of the structural conformation and the biological activity of BMOG comprising growing, under suitable culture conditions, said hosts cells transformed or transfected with a DNA molecule of claim 1 in a manner allowing expression of such polypeptide product, and recovering said product.

8. A polypeptide having an amino acid sequence which comprises BMOG.

9. A polypeptide according to claim 8 said amino acid sequence comprising SEQ ID NO 4, SEQ ID NO 5, or SEQ ID NO 6, or a variant thereof.

10. The polypeptide of claim 8 wherein said polypeptide is soluble.

11. An IgG fusion protein comprising BMOG or a fragment thereof.

12. An antibody to a polypeptide of claim 8, 9, 10 or 11.

13. A hybridoma cell line which produces an antibody specific to BMOG.

14. A method for modulating the immune system of a subject comprising administering to the subject a therapeutically effective amount of a BMOG polypeptide or a fragment thereof.

15. The method of claim 14 wherein said polypeptide is soluble.

16. The method of claim 14 wherein said polypeptide is a fusion protein.

17. The method of claim 14 wherein the subject is human.

18. A method of inhibiting signal transduction involving a cell expressing BMOG comprising contacting said cell with a soluble BMOG protein.

19. A method for targeting a toxin, imageable compound or radionuclide to a cell expressing BMOG comprising contacting the cell with a BMOG protein, fragment, or fusion protein thereof.

20. A method of gene therapy comprising the step of administering a gene for BMOG to a subject.


SEQUENCE LISTING (20 sequences)

SEQ ID NO: 1 - DNA, 671 bp, Homo sapien
gtccttcctcctccacccagacctcactgctcagatccccttcgccaactgggacatcttccgacatggcctggatgctgttgctcatcttgatcatggtccatccaggatcctgtgctctctgggtgtcccagccccctgagattcgtaccctggaaggatcctctgccttcctgccctgctccttcaatgccagccaagggagactggccattggctccgtcacgtggttccgagatgaggtggttccagggaaggaggtgaggaatggaaccccagagttcaggggcgcctggccccacttgcttcttcccgtttcctccatgaccaccaggctgagctgcacatccgggacgtgcgaggccatgacgccagcatctacgtgtgcagagtggaggtgctgggccttggtgtcgggacagggaatgggactcggctggtggtggagaaagaacatcctcagctaggggctggtacagtcctcctccttcgggctggattctatgctgtcagctttctctctgtggccgtgggcagcaccgtctattaccagggcaaatgccactgtcacatgggaacacactgccactcctcagatgggccccgaggagtgattccagagcccagatgtccctagtcctcttcaaaagaccccaataaatctgccccac

SEQ ID NO: 2 - DNA, 889 bp, Homo sapien
gtccttcctcctccacccagacctcactgctcagatccccttcgccaactgggacatcttccgacatggcctggatgctgttgctcatcttgatcatggtccatccaggatcctgtgctctctgggtgtcccagccccctgagattcgtaccctggaaggatcctctgccttcctgccctgctccttcaatgccagccaagggagactggccattggctccgtcacgtggttccgagatgaggtggttccagggaaggaggtgaggaatggaaccccagagttcaggggcgcctggccccacttgcttcttcccgtttcctccatgaccaccaggctgagctgcacatccgggacgtgcgaggccatgacgccagcatctacgtgtgcagagtggaggtgctgggccttggtgtcgggacagggaatgggactcggctggtggtggagaaagaacatcctcagctaggggctggtacagtcctcctccttcgggctggattctatgctgtcagctttctctctgtggccgtgggcagcaccgtctattaccagggcaaatatgccaaatctactctctccggattcccccaactctgaactttcccttccaccaggtctgacctggaaaggtccaagaaggcagctgccggctgtggtcccagcgcccctcccaccaccatgtgggagctcagcacatctgcttcccccagtcccaggaggctgagcctgattgtcctgagaaatgggaaggatcagatatgactcctccttggcaactgccctttcctgccaggcccacacataccctcttctggctgttaggggagcttgggtccctgaacactgtcattcacccaacaaattactatttgaccccagagtgggtgg

SEQ ID NO: 3 - DNA, 834 bp, Homo sapien
gtccttcctcctccacccagacctcactgctcagatccccttcgccaactgggacatcttccgacatggcctggatgctgttgctcatcttgatcatggtccatccaggatcctgtgctctctgggtgtcccagccccctgagattcgtaccctggaaggatcctctgccttcctgccctgctccttcaatgccagccaagggagactggccattggctccgtcacgtggttccgagatgaggtggttccagggaaggaggtgaggaatggaaccccagagttcaggggcgcctggccccacttgcttcttcccgtttcctccatgaccaccaggctgagctgcacatccgggacgtgcgaggccatgacgccagcatctacgtgtgcagagtggaggtgctgggccttggtgtcgggacagggaatgggactcggctggtggtggagaaagaacatcctcagctaggggctggtacagtcctcctccttcgggctggattctatgctgtcagctttctctctgtggccgtgggcagcaccgtctattaccagggcaaatgtctgacctggaaaggtccaagaaggcagctgccggctgtggtcccagcgcccctcccaccaccatgtgggagctcagcacatctgcttcccccagtcccaggaggctgagcctgattgtcctgagaaatgggaaggatcagatatgactcctccttggcaactgccctttcctgccaggcccacacataccctcttctggctgttaggggagcttgggtccctgaacactgtcattcacccaacaaattactatttgaccccagagtgggtgg

SEQ ID NO: 4 - protein, 190 aa, Homo sapien
MAWMLLLILIMVHPGSCALWVSQPPEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLVVEKEHPQLGAGTVLLLRAGFYAVSFLSVAVGSTVYYQGKCHCHMGTHCHSSDGPRGVIPEPRCP

SEQ ID NO: 5 - protein, 177 aa, Homo sapien
MAWMLLLILIMVHPGSCALWVSQPPEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLVVEKEHPQLGAGTVLLLRAGFYAVSFLSVAVGSTVYYQGKYAKSTLSGFPQL

SEQ ID NO: 6 - protein, 201 aa, Homo sapien
MAWMLLLILIMVHPGSCALWVSQPPEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLVVEKEHPQLGAGTVLLLRAGFYAVSFLSVAVGSTVYYQGKCLTWKGPRRQLPAVVPAPLPPPCGSSAHLLPPVPGG

SEQ ID NO: 7 - protein, 185 aa, Homo sapien
MAWMLLLILIMVHPGSCALWVSQPPEIRTEGSSAFLPCSFNASQGRLAIGSVTWFRDEVVPGKEVRNGTPEFRGRLAPLASSRFLHDHQAELHIRDVRGHDASIYVCRVELGLGVGTGNGTRLVVEKEHPQLGAGTVLLLRAGFYAVSFLSVAVGSTVYYHGKCHCHMGTHCHSSDGVIPEPRCP

SEQ ID NO: 8 - protein, 175 aa, Homo sapien
MAWMLLLILIMVHPGSCALWVSQPPEIRTEGSSAFLPCSFNASQGRLAIGSVTWFRDEVVPGKEVRNGTPEFRGRLAPLASSRFLHDHQAELHIRDVRGHDASIYVCRVELGLGVGTGNGTRLVVEKEHPQLGAGTVLLLRAGFYAVSFLSVAVGSTVYYHGKYAKSTLSGFPQL

SEQ ID NO: 9 - protein, 198 aa, Homo sapien
MAWMLLLILIMVHPGSCALWVSQPPEIRTEGSSAFLPCSFNASQGRLAIGSVTWFRDEVVPGKEVRNGTPEFRGRLAPLASSRFLHDHQAELHIRDVRGHDASIYVCRVELGLGVGTGNGTRLVVEKEHPQLGAGTVLLLRAGFYAVSFLSVAVGSTVYYHGKCLTWKGPRRLPAVVPAPLPPPCGSSAHLLPPVPGG

SEQ ID NO: 10 - protein, 161 aa, Homo sapien
MAWMLLLILIMVHPGSCALWVSQPPEIRTLEGSSAFLPCSFNASQGRLAIGSVTWFRDEVVPGKEVRNGTPEFRGRLAPLASSRFLHDHQAELHIRDVRGHDASIYVCRVELGLGVGTGNGTRLVVEKEHPQLGAGTVLLLRAGFYAVSFLSVAVGSTVYY

SEQ ID NO: 11 - protein, 185 aa, Homo sapien
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVEDPFYWVSPGVLVLLAVLPVLLLQITVGLVFLCL

SEQ ID NO: 12 - protein, 175 aa, Rat
MAGVWSLSLPSCLLSLLLLLQLSRSYAGQFRVIGPGHPIRALVGDEAELPCRISPGKNATCMEVGWYRSPFSRVVHLYRNGKDQDAEQAPEYRGRTELLKESIGEGKVALRIQNVRFSDEGGYTCFFRDHSYQEEAAVELKVEDPFYWINPGVLALIALVPMLLLVSVGLVFLFL

SEQ ID NO: 13 - protein, 175 aa, Mouse
MACLWSFSWPSCFLSLLLLLLLQLSCSYAGQFRVIGPGYPIRALVGDEAELPCRISPGKNATGMEVGWYRSPFSRVVHLYRNGKDAEQAPEYRGRTELLKETISEGKVTLRIQNVRFSDEGGYTCFFRDHSYQEEAAMELKVEDPFYWVNPGVLTLIALVPTILLVSVGLVFLFL

SEQ ID NO: 14 - DNA, 90 bp, Chicken
  misc_feature (1)...(90): n = A,T,C or G
matsgcnhstwrtyvahgsatvvasrvtavgdvvrchsckdvrnsdrwrssrvhhyrngvdgmykgrtrdgsdgndrtavtssdsgsysc

SEQ ID NO: 15 - protein, 123 aa, Homo sapien
MASSLAFLLLNFHVSLLLVQLLTPCSAQFSVLGPSGPILAMVGEDADLPCHLFPTMSAETMELKWVSSLRQVVNVYADGKEVEDRQSAPYRGRTSILRDGITAGKAALRIHNVTASDSGQLEC

SEQ ID NO: 16 - protein, 124 aa, Bovine
MAVFPNSCLAGCLLIFILLQLPKLDSAPFDVIGPPEPILAVVGEDAELPCRLSPNVSAKGMELRWFREKVSPAVFVSREGQEQEGEEMAEYRGRVSLVEDHIAEGSVAVRIQEVKASDDGEYRC

SEQ ID NO: 17 - protein, 116 aa, Homo sapien
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYEC

SEQ ID NO: 18 - protein, 104 aa, Homo sapien
MGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQC

SEQ ID NO: 19 - DNA, 34 bp, Homo sapien
aactgcagcggccgccatggcctggatgctgttg

SEQ ID NO: 20 - DNA, 58 bp, Homo sapien
atagtttagcggccgctcagtgatggtggtgatggtggtcgactgtaccagcccctag